(19) United States
(12) Patent Application Publication
(10) Pub. No.: US 2024/0229159 A1
(43) Pub. Date: Jul. 11, 2024

(54) GENE SIGNATURE FOR THE IDENTIFICATION OF LYMPH NODE INVOLVEMENT IN CANCER PATIENTS

(71) Applicant: University of Limerick, Limerick (IE)

(72) Inventors: Colum Dunne, Limerick (IE); Patrick Kiely, Limerick (IE); Joanne Nolan, Limerick (IE); Ailish Hannigan, Limerick (IE); Shona Tormey, Limerick (IE); Anne Merrigan, Limerick (IE); Mary McCumiskey, Limerick (IE)

(21) Appl. No.: 18/573,842

(22) PCT Filed: Jun. 30, 2022
(86) PCT No.: PCT/EP2022/068191

§ 371 Date: Dec. 22, 2023

(30) Foreign Application Priority Data
Jun. 30, 2021 (EP) 21182896.7

(51) Int. Cl. C12Q 1/6886; G16B 25/10

(52) U.S. Cl. CPC C12Q 1/6886; G16B 25/10; C12Q 2600/112; C12Q 2600/118; C12Q 2600/158

(57) ABSTRACT

A method for determining the presence or absence of lymph node metastasis in a breast cancer subject is provided. The method comprises the steps of assaying for expression of genes (or proteins encoded by the genes) ITGB4 and SNAI2 in a sample from the subject, wherein an increased expression of ITGB4 and a decreased expression of SNAI2 compared with the expression of the same genes (or proteins encoded by said genes) in a subject without cancer is indicative of the absence of lymph node metastasis in said subject. or wherein an increased expression of SNAI2 and a decreased expression of ITGB4 compared with the expression of the same genes (or proteins encoded by said genes) in a subject without cancer is indicative of the presence of lymph node metastasis in said subject.

### FIELD OF THE INVENTION

The current invention relates to the identification of lymph node involvement in cancer patients, especially breast cancer patients, utilising a gene signature comprising ITGB4 and SNAI2. The current invention further relates to the prediction of prognosis in breast cancer patients.

### BACKGROUND OF THE INVENTION

Metastasis is the most perilous aspect of cancer, characterised by the ability for a cell to become autonomous from its host and colonise in a secondary site. It remains the only attribute, which distinguishes malignant cancer from a benign growth. Metastasis requires a series of delicate, intricate and complex set of sequential steps often referred to as the metastatic cascade. We have come to learn that not all cells which create a tumour have the capacity to undergo every step in the cascade and so will not colonise at secondary sites. These elaborate stages have recently been defined by Welch and Hurst to include: motility, modulation of the microenvironment, plasticity and colonisation (Welch, D. R. et al., Defining the Hallmarks of Metastasis. Cancer research, 2019. 79(12): p. 3011-3027).

Metastasis is the major cause of death among breast cancer patients and the most common site of metastasis for these patients is the brain, bone, liver and lungs (Lu, X. and Y. Kang, Organotropism of breast cancer metastasis. J Mammary Gland Biol Neoplasia, 2007. 12(2-3): p. 153-62). Furthermore, breast cancer remains the most commonly invasive cancer among women worldwide.

The current staging system for breast cancer patients involves assessing tumour size, the presence or absence of metastatic spread to the lymph nodes and the presence or absence of distant metastasis. Often, the lymph node is one of the first sites of metastatic spread before invasion into other organs. A lymph node biopsy is a vital indicator of cancer spread and crucial for the staging of breast cancer. This process often involves an ultrasound guided biopsy or resecting the sentinel lymph nodes during surgery for pathological evaluation. Ultrasound guided biopsy is recommended if axillary lymph nodes appear suspicious with a cortical thickness ≥3 mm and if axillary nodes appear suspicious upon examination, but appear clinically normal upon imaging (NICE, locally advanced breast Cancer: full guideline. Cardiff: National Collaborating Centre for Cancer, 2009). However, imaging and examination alone are not sufficient to rule out the possibility of nodal involvement for cancer staging. Interestingly there is currently no dependable method that can be used to diagnose lymph node involvement preoperatively. Therefore, a sentinel lymph node biopsy is recommended even if ultrasound and physical examination of the region appear normal. It is considered that women whose sentinel biopsy results identify macrometastasis (>2 mm) should have the possibility of axillary dissection discussed at the clinics multidisciplinary meetings. Axillary node dissections can be complex procedures which have the potential for side effects such as lymphedema, wound infection and tenderness. Therefore, this procedure is decided upon based on a patient's clinical presentation, tumour burden and presence or absence of nodal involvement. As the lymph nodes connect to our blood stream through the efferent lymphatics this provides a direct route for cancer cells to circulate through the body.

Breast cancer is a prime example of how successful gene expression tools can be applied in the clinic to aid in decision-making. Molecular approaches used for the characterisation and stratification of breast cancers have revolutionised breast cancer treatment. The identification of hormone receptors allows for a personalised therapeutic approach for breast cancer patients (Lippman, M. E. et al., Oestrogen-responsive human breast cancer in long term tissue culture. Nature, 1975. 256(5518): p. 592-3). Classified predictive biomarkers are applied clinically for the molecular identification of patients who have a higher risk of relapse.

MammaPrint™, a diagnostic tool consisting of 70 genes, was the first commercially available gene expression test to predict the likelihood of metastasis for breast cancer patients (Cardoso, F., et al., 70-gene signature as an aid to treatment decisions in early-stage breast cancer. New England Journal of Medicine, 2016. 375(8): p. 717-729). The Oncotype DX™ is a 21 gene signature used clinically to predict breast cancer recurrence and to identify women who will gain clinical benefit from adjuvant chemotherapy (Paik, S., et al., Gene expression and benefit of chemotherapy in women with node-negative, estrogen receptor-positive breast cancer. J Clin Oncol, 2006. 24(23): p. 3726-3734). The OncoMasTR® is a three gene signature with the ability to predict distant recurrence in women with ER+ breast cancer (Kelly, C. M., et al., Comparison of the prognostic performance between OncoMasTR and OncotypeDX multigene signatures in hormone receptor-positive, HER2-negative, lymph node-negative breast cancer. 2018, American Society of Clinical Oncology).

Many studies utilising gene expression platforms have identified variation in expression patterns between LN− and LN+ tissue samples (Bertucci, F. o., et al., Human Molecular Genetics, 2000. 9(20): p. 2981-2991; Abba, M. C., et al., Mol Cancer Res, 2007. 5(9): p. 881-90; Huang, E., et al., The Lancet, 2003. 361(9369): p. 1590-1596). Further studies have attempted to interrogate patterns of dysregulation but have failed to result in clinical utility for the predicative identification of LN+ patients (Lu et al., Breast Cancer Res Treat, 2008. 108(2):p. 191-201). Most notably, a gene expression study by Ellsworth et al showed a 90% correct classification of LN− samples however, clustering analysis was only successful in classifying 66% of LN+ samples (Ellsworth, R. E., et al., International Journal of Breast Cancer, 2011. 2011: p. 142763). Some papers have even noted that the addition of gene expression hindered classification models which may be due to confounding factors such as tumour heterogeneity, cell plasticity and the effect of the tumour microenvironment on total gene expression analysis.

WO2006032769 discloses a method of determining whether a breast cancer has invaded the lymph nodes. The method utilizes a biochip. The probes correspond to genes expression levels of which are modified in mammary tumours, thus allowing the study of parameters, such as lymph node invasion, among others. Although node involvement is a critical prognostic indicator in breast cancer, gene expression tools have yet to be successful for the preoperative identification of lymph node negative (LN) or lymph node positive (LN+) breast cancer patients.

Castillo-Lluva S et al., (Castillo-Lluva S et al, A new role of SNAI2 in postlactational involution of the mammary gland links it to luminal breast cancer development, Oncogene, Nature publishing group UK, vol. 34, no. 36, 22 June 2015) discloses that SNAI2 is important in the development of breast cancer of luminal origin.

Ruan Shasha et al., (Ruan Shasha et al, integrin [beta]4-targeted cancer immunotherapies inhibit tumor growth and decrease metastasis, Cancer Research, vol. 80, no. 4, 15 February 2020) discusses integrin β4 (ITGB4) role in the regulation of cancer stem cells (CSC). It discusses that upregulation of ITGB4 is seen with lung metastasis and it is an adverse prognostic marker in pancreatic ductal adenocarcinoma and breast cancer. The authors discuss the targeting of ITGB4 as an approach with clinical benefit.

Vouriluoto K et al., (Vouriluoto K et al., Vimentin regulates EMT induction by Slug and oncogenic H-Ras and migration by governing Axl expression in breast cancer, Oncogene, vol. 30, no. 12, 1 March 2011) reports that ectopic expression of oncogenic H-Ras-V12G and Slug (SNAI2) induces vimentin expression and migration in pre-malignant breast epithelial cells. Vimentin is a marker of epithelial-to-mesenchymal transition (EMT) and the authors conclude that Slug and Ras-induced EMT changes are dependent on the upregulation of vimentin.

The current invention serves to alleviate the problems of the prior art by providing a method to predict preoperative identification of lymph node status in a breast cancer patient. This method provides a dependable tool for the preoperative identification of lymph node involvement, creating opportunity for earlier interventions that may prove imperative in the fight against metastasis. By preoperatively identifying lymph node involvement, this allows for a more accurate identification of patients who require lymph node sampling or dissection, reducing the potential side effects associated with these procedures. This also unlocks the prospect of more accurately identifying those who would benefit from neoadjuvant therapy, namely neoadjuvant radiotherapy to the axilla, in a bid to prevent further cancer cell migration.

### SUMMARY OF THE INVENTION

The axillary lymph nodes are often the first sites of metastatic spread before invasion into other organs. Therefore, lymph node status is a vital indicator of cancer spread and crucial for the staging of breast cancer. Currently, imaging and physical examination alone are not sufficient to rule out the possibility of nodal involvement for cancer staging. There exists a current lack of prospective molecular tools for the earlier identification of lymph node involvement. Despite these advancements in molecular diagnostics, there remains no clinically relevant molecular biomarkers capable of indicating whether a cancer has spread, or is more likely to spread, to the lymph nodes.

The current invention provides a gene signature to discriminate between lymph node positive (LN+) and lymph node negative (LN−) breast cancer patients. The preoperative identification of lymph node positive patients could allow for the delivery of neoadjuvant therapy, targeting processes that may result in distant metastasis.

The current method has been tested and validated using patent tissue samples and can be performed on a patient's initial core biopsy. This signature, optionally in conjunction with current methods of lymph node examination, could aid in the swift identification of lymph node positive/negative breast cancer patients.

The use of patient grade, routinely examined at time of biopsy, and genes ITGB4 and SNAI2 correctly classified lymph node status for 73% of patient samples. Increasing expression of ITGB4 was associated with a decrease in the odds of lymph node positive status (odds ratio (OR): 0.53, 95% confidence interval (CI): 0.28-1.01). Increasing expression of SNAI2 was associated with an increase in the odds of lymph node positive status (OR: 1.7, 95% CI: 1.0-3.12).

An aspect of the current invention provides a method for determining the presence or absence of lymph node metastasis, or the risk of lymph node metastasis, in a cancer subject, the method comprising,
assaying for expression of one or more of genes (or one or more proteins encoded by said genes) selected from ITGB4 and SNAI2 in a sample from said subject,
wherein an increased expression of SNAI2 and/or decreased expression of ITGB4, compared with the expression of said gene(s) (or protein(s) encoded by said gene(s)) in a subject without cancer is indicative of the presence of lymph mode metastasis in the subject,
or wherein an increased expression of ITGB4 and/or a decreased expression of SNAI2, compared with the expression of said gene(s) (or protein(s) encoded by said gene(s)) in a subject without cancer is indicative of the absence of lymph node metastasis in the subject.

In an embodiment, an increased expression of SNAI2 and a decreased expression of ITGB4, compared with the expression of said genes (or proteins encoded by said genes) in a subject without cancer is indicative of the presence of lymph mode metastasis in the subject, or wherein an increased expression of ITGB4 and a decreased expression of SNAI2 compared with the expression of said genes (or proteins encoded by said genes) in a subject without cancer is indicative of the absence of lymph node metastasis in the subject.

Preferably, said cancer is breast cancer.

Preferably, a hormone receptor (HR) positive breast cancer.

Preferably, the sample is a tumour biopsy sample. Typically, such a sample is a core biopsy, such as a core punch biopsy.

Preferably, the sample is one from the primary breast tumour.

Preferably the lymph node is selected from one or more of a sentinel lymph node and an axillary lymph node.

In an embodiment, the method further comprises determining the grade of tumour present in the subject.

An aspect of the invention provides a method for staging a cancer in a subject, preferably a breast cancer. A test suggestive of metastasis or a test result suggestive of no metastasis informs staging based on likelihood of distant cancer. The method comprising,
assaying for expression of one or more of genes (or proteins encoded by said genes) selected from ITGB4 and SNAI2 in a sample from said subject to determine the presence or absence of lymph node metastasis in the subject,
wherein an increased expression of SNAI2 and/or decreased expression of ITGB4, compared with the expression of said gene(s) (or protein(s) encoded by said gene(s)) in a subject without cancer is indicative of the presence of lymph mode metastasis in the subject,
or wherein an increased expression of ITGB4 and/or a decreased expression of SNAI2, compared with the expression of said gene(s) (or protein(s) encoded by said gene(s)) in a subject without cancer is indicative of the absence of lymph node metastasis in the subject.
and determining the stage of a cancer based on the presence or absence of lymph node metastasis in the subject.

Preferably, wherein an increased expression of SNAI2 and a decreased expression of ITGB4, compared with the expression of said genes (or proteins encoded by said genes) in a subject without cancer is indicative of the presence of lymph mode metastasis in the subject, or wherein an increased expression of ITGB4 and a decreased expression of SNAI2 compared with the expression of said genes (or proteins encoded by said genes) in a subject without cancer is indicative of the absence of lymph node metastasis in the subject.

An aspect of the invention provides a method for predicting risk of metastasis in a subject with cancer, preferably breast cancer, the method comprising,
assaying for expression of one or more of genes (or proteins encoded by said genes) selected from ITGB4 and SNAI2 in a sample from said subject,
wherein an increased expression of SNAI2 and/or a decreased expression of ITGB4, compared with the expression of said gene(s) (or protein(s) encoded by said gene(s)) in a subject without cancer is indicative of the presence of lymph mode metastasis in the subject
or wherein an increased expression of ITGB4 and/or a decreased expression of SNAI2 compared with the expression of said gene(s) (or protein(s) encoded by said gene(s)) in a subject without cancer is indicative of the absence of lymph node metastasis in the subject,
and determining the risk based on the presence or absence of lymph node metastasis in the subject.

Preferably, wherein an increased expression of SNAI2 and a decreased expression of ITGB4, compared with the expression of said genes (or proteins encoded by said genes) in a subject without cancer is indicative of the presence of lymph mode metastasis in the subject, or wherein an increased expression of ITGB4 and a decreased expression of SNAI2 compared with the expression of said genes (or proteins encoded by said genes) in a subject without cancer is indicative of the absence of lymph node metastasis in the subject.

Typically, metastasis is distant metastasis.

A further aspect of the current invention provides a method for treating cancer in a subject, preferably breast cancer, said method comprising,
assaying for expression of one or more genes selected from (or proteins encoded by said genes) selected from ITGB4 and SNAI2 in a sample from said subject to determine the presence or absence of lymph node metastasis as per the method of the invention,
classifying said subject as having lymph node metastasis or absence of lymph node metastasis based on the expression,
administering cancer treatment to said subject based on the classification.

Typically, the treatment is neo-adjuvant treatment.

Typically, the subject will then undergo a surgical treatment to remove the tumour or part thereof and/or lymph nodes, or part thereof.

A method of identifying a cancer subject, preferably breast cancer subject, that is suitable for treatment, the method comprising a step of assaying for expression of one or more of genes (or proteins encoded by said genes) ITGB4 and SNAI2 in a sample from said subject,
wherein an increased expression of SNAI2 and a decreased expression of ITGB4, compared with the expression of said genes (or proteins encoded by said genes) in a subject without cancer is indicative of the presence of lymph mode metastasis in the subject and that the subject is suitable for treatment.

Typically, the treatment is neo-adjuvant treatment.

Preferably, the subject will then undergo a surgical treatment to remove the tumour or part thereof and/or lymph nodes, or part thereof.

A further aspect of the current invention provides a method for determining a treatment plan for a subject with cancer, preferably breast cancer, said method comprising,
assaying for expression of one or more genes selected from (or proteins encoded by said genes) selected from ITGB4 and SNAI2 in a sample from said subject to determine the presence or absence of lymph node metastasis as per the method of the invention,
classifying said subject as having lymph node metastasis or absence of lymph node metastasis based on the expression,
determining the treatment plan based on the classification.

Typically, the treatment is neo-adjuvant treatment.

Preferably, the subject will then undergo a surgical treatment to remove the tumour or part thereof and/or lymph nodes, or part thereof.

In one aspect of the invention, there is provided a system for obtaining data from at least one test sample obtained from at least one individual, the system comprising:
a determination module configured to receive at least one test sample and perform at least one test analysis on the test sample to assay for expression of at one or more of ITGB4 and SNAI2,
optionally, a storage system for storing expression data generated by the determination module; and
a display module for displaying a content based in part on the data output from said determination module, wherein the content comprises a signal indicative of the expression of the genes.

### BRIEF DESCRIPTION OF THE FIGURES

The current invention will now be described with reference to the following Figures in which;

FIG. 1: Heatmaps showing the intensity of dysregulation across breast cancer tissue samples. A) Breast cancer samples compared to normal tissue and stratified into their respective stages. The spread around the mean for the normal tissue is displayed. B) Node positive breast cancer samples compared to cancer tissue from patients with no node involvement. The spread around the mean for those with no node involvement is also displayed.

FIG. 2 Spearman's correlations across node positive and node negative tissue samples (n=22): Heatmap showing the association of expression between genes in patient tissue samples using Spearman's correlation coefficient

FIG. 3: Kaplan-Meier plots analysing the expression of ITGB4 (A) and SNAI2 (B) in distant metastasis free survival among a population of breast cancer patients (n=191). KM plots showing distant metastasis free survival for low and high mRNA expression of ITGB4 and SNAI2. Included were all ER+and PR+patients with a combination of HER+/−

FIG. 4: Kaplan-Meier plots analysing the expression of ITGB4 (A) AND SNAI2 (B) in distant metastasis free survival among hormone positive breast cancer patients (n=47). KM plots showing distant metastasis free survival for low and high mRNA expression of ITGB4 and SNAI2. Included were all ER+ and PR+ and HER+ patients.

FIG. 5: Lymph node diagnosis timeline. A representative image of how dependable preoperative lymph node diagnosis could influence neoadjuvant therapy and further impact the timeline of adjuvant therapy.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

The current invention provides a method for predicting or determining the lymph node involvement in a breast cancer patient. The method comprises determining the expression of a gene signature. The signature comprises one or more genes selected from ITGB4 and SNAI2. Preferably, the genes selected are both ITGB4 and SNAI2.

The expression of the selected gene(s) is determined in a sample from the subject. Preferably that sample is a cancer biopsy., preferably, a breast cancer biopsy.

Notably, the sample is one from the primary tumour, typically a core biopsy. Typically, such a sample is a core punch biopsy. The biopsy is usually one taken during the clinical process of identifying and/or staging the cancer. The biopsy may be, but not limited to, a fine needle aspiration biopsy, stereotactic biopsy, core needle biopsy or open surgical biopsy. Methods of obtaining core biopsies from tumours are known in the art.

In an embodiment, determining increased expression of ITGB4 and a decreased expression of SNAI2 compared with expression of the same gene(s) in a subject without cancer is indicative of no lymph node metastasis in said subject. Such a patient would be classified as L−. These results are surprising or unexpected and contradictory in comparison to the literature pertaining to the role of the B4 subunit in breast cancer. A study by Lam et al (Cancer Treatment Reviews, 2014, 40(1):p. 129-138.5) suggest that an increased expression of ITGB4 decreases the likelihood of lymph node involvement and cancer metastasis.

Notably, determining an increased expression of SNAI2 and a decreased expression of ITGB4 compared with expression of the same gene(s) in a subject without cancer is indicative of lymph node metastasis in said subject. Such a patient would be classified as LN+.

The invention also encompasses any of the methods disclosed herein wherein the sensitivity is 50% or more, 60% or more, 70% or more, 80% or more, 90% or more or 95% or more. The invention also encompasses any of the methods disclose herein wherein the specificity is 50% or more, 60% or more, 70% or more, 80% or more, 90% or more or 95% or more. Preferably, the sensitivity is at least 70% and the specificity is at least 70%. Preferably, the sensitivity is at least 80% and the specificity is at least 80%. Preferably, the sensitivity is at least 70% and the specificity is at least 70%. Preferably, the sensitivity is at least 90% and the specificity is at least 90%.

In an embodiment, a 1.5 fold increase in expression of SNAI2 is indicative of lymph node metastasis in said subject. It may be 1.5 fold or more, 2 fold or more, 2.5 fold or more, 3 fold or more, or 3.5 fold or more, 4 fold or more, 4.5 fold or more, or 5 fold or more. Alternatively, the increased expression may be 10% or more, 20% or more, 30% or more, 40% or more, 50% or more, 60% or more.

In an embodiment, a 1.5 fold decrease in expression of ITGB4 is indicative of lymph node metastasis in said subject. It may be 1.5 fold or more, 2 fold or more, 2.5 fold or more, 3 fold or more, or 3.5 fold or more, 4 fold or more, 4.5 fold or more, or 5 fold or more. Alternatively, the decreased expression may be 10% or more, 20% or more, 30% or more, 40% or more, 50% or more, 60% or more.

For example, in an embodiment, a 1.5 fold or more increase in expression of SNAI2 and a 1.5 fold decrease or more in expression of ITGB4 is indicative of lymph node metastasis in said subject tested.

It will be appreciated that any one of the noted increases in expression of SNAI2 as disclosed above maybe combined with any one of the noted decreases in expression of ITGB4.

In an embodiment, a 1.5 fold increase in expression of ITGB4 is indicative of no lymph node metastasis in said subject. It may be 1.5 fold or more, 2 fold or more, 2.5 fold or more, 3 fold or more, or 3.5 fold or more, 4 fold or more, 4.5 fold or more, or 5 fold or more. Alternatively, the increased expression may be 10% or more, 20% or more, 30% or more, 40% or more, 50% or more, 60% or more.

In an embodiment, a 1.5 fold decrease in expression of SNAI2 is indicative of no lymph node metastasis in said subject. It may be 1.5 fold or more, 2 fold or more, 2.5 fold or more, 3 fold or more, or 3.5 fold or more, 4 fold or more, 4.5 fold or more, or 5 fold or more. Alternatively, the decreased expression may be 10% or more, 20% or more, 30% or more, 40% or more, 50% or more, 60% or more.

For example, a 1.5 fold or more increase in expression of ITGB4 and a 1.5 fold or more decrease in expression of SNAI2 is indicative of no lymph node metastasis in said subject tested.

It will be appreciated that any one of the noted increases in expression of ITGB4 as discussed above maybe combined with any one of the noted decreases in expression of SNAI2.

The ITGB4 gene codes for integrin subunit β4 and the β4 subunit is unusual in that it partners solely with the α6 subunit (Hynes, R. O., Cell, 2002. 110(6): p. 673-687). The physiological role of the β4 subunit involves connecting the breast epithelial to the basement membrane. The β4 subunit has been shown to be involved in cell proliferation, invasion and migration; moreover, knock out of the β4 subunit suppresses breast cancer tumorigenesis (Falcioni, R., et al., Exp Cell Res, 1997. 236(1): p. 76-85). Furthermore, the overexpression of β4 is linked with the more aggressive basal like breast cancer subtype (Lam et al., Cancer Treatment Reviews, 2014. 40(1): p. 129-138.5). An exemplary sequence of ITGB4 and the protein it encodes can be found at NCBI Gene ID 3691. It will be appreciated that variants of the gene sequence (or the protein sequence), including fragments, may occur and all such may be included in the method of the invention. Primers, probes and antibodies are also known in the art.

The SNAI2 gene, also referred to as SLUG, is a member of snail family, and codes for the Snai2 protein, a zinc finger transcription factor. These conserved zinc finger proteins are well regarded for their role as epithelial to mesenchymal transition (EMT) transcription factors (Zhou, W., et al,. Journal of Cell Science, 2019. 132(23)). The translated Snai2 protein is primarily known for its role in EMT during developmental processes. An exemplary sequence of SNAI2 and the protein it encodes can be found at NCBI ID 6591. It will be appreciated that variants of the gene sequence (or the protein sequence), including fragments, may occur and all such may be included in the method of the invention. Primers, probes and antibodies are also known in the art.

The method may further comprise determining the grade of the tumour present in the subject. Methods of determining the grade are well known in the art. The implications of grading relate to choices made regarding requirement for, and type of, diagnostic investigation (e.g., imaging), choice of treatment approach and potentially prognosis.

An aspect of the invention provides a method for predicting prognosis in a breast cancer patient. If the subject is determined to have no lymph node involvement or metastasis as per the method of the invention, this is indicative of a good prognosis. If the subject is determined to have lymph node involvement, this is indicative of a poor prognosis. Poor prognosis can be a decrease in the likelihood of survival compared to a good prognosis.

The method of the current invention may be one to predict the risk of metastasis in a subject, particularly distant metastasis. In the method if a subject determined to have an increased expression of ITGB4, and a decreased expression of SNAI2, the subject is predicted to have no lymph node involvement or metastasis. This is indicative of a low risk of metastasis or distant metastasis elsewhere in the body, e.g. in the brain, bone, liver, and/or lungs.

If a subject is determined to have an increased expression of SNAI2 and a decreased expression of ITGB4 the subject is predicted to have lymph node involvement. This is indicative of a high risk of distant metastasis elsewhere in the body, e.g. in the brain, bone, liver, and/or lungs.

A further aspect of the current invention provides a method for treating breast cancer in a subject, said method comprising,
assaying for expression of one or more genes selected from (or proteins encoded by said genes) ITGB4 and SNAI2 in a sample from said subject to determine the presence or absence of lymph node metastasis in the subject as per the method of the invention,
determining and/or providing a suitable treatment to said subject based on the presence or absence of lymph node metastasis in the subject.

Notably, an increased expression of SNAI2 and a decreased expression of ITGB4, compared with the expression of said genes (or proteins encoded by said genes) in a subject without cancer is indicative of the presence of lymph mode metastasis in the patient.

This subject may then undergo lymph node sampling or dissection. This sampling may be ultrasound guided biopsy of the lymph modes. This sampling or dissection may be carried out to confirm the presence of lymph node metastasis in the subject and/or to determine the extent of involvement. These procedures have numerous potential side effects, and the method of the invention helps to avoid or reduce the number of unnecessary procedures being undertaken.

These subjects may then undergo further therapy, such as neoadjuvant or adjuvant therapy. This is primarily to the axilla in a bid to prevent further cancer cell migration. However, other therapeutic approaches may be chosen as appropriate, based on available products and advances in oncology therapeutics (e.g., biologics, antibodies, precision medicine treatments).

The neoadjuvant therapy may be a hormone treatment and/or chemotherapy treatment prior to surgery. This may be any suitable breast cancer treatment. Such treatments are known in the art. The treatments may include but are not limited to anthracycline or taxane based therapies. For examples, for HER2 positive tumours, neoadjuvant therapy may include a combination of chemotherapy and HER2 targeted drugs such as Herceptin (trastuzumab). The subject may also, or alternatively, undergo a lymph node removal or partial lymph node removal. This may be at the same time as the initial surgery of the tumour. The subject may then undergo a surgical treatment to remove or partially remove the tumour.

An increased expression of ITGB4 and a decreased expression of SNAI2 compared with the expression of said genes (or proteins encoded by said genes) in a subject without cancer, is indicative of the absence of lymph node metastasis in the patient. Typically, if the patient is negative the treatment plan is then dependent upon patient-specific parameters such as hormonal status, tumour size etc. There are several other factors which dictate the treatment approach, and this would be known to a person skilled in the art.

The invention also provides a method of identifying a subject that is suitable for treatment or a method for determining a treatment plan for a subject with cancer. Both methods comprise determining the presence or absence of lymph node metastasis as per the method of the invention.

In the method of identifying a subject that is suitable for treatment, an indication of the presence of lymph node metastasis in the subject indicates that the subject is suitable for treatment. This treatment may be neo-adjuvant treatment. In addition, or alternatively, the treatment may be a surgical treatment to remove the tumour or part thereof and/or lymph nodes, or part thereof.

The treatment may be lymph node sampling or dissection. This sampling may be ultrasound guided biopsy of the lymph modes. This sampling or dissection may be carried out to confirm the presence of lymph node metastasis in the subject and/or to determine the extent of involvement.

In the method for determining the treatment plan for a subject, the treatment may be any treatment as disclosed herein in relation to the methods of the invention.

For example, if the subject is determined to have lymph node metastasis, the treatment plan may be lymph node sampling or dissection, optionally followed by neo-adjuvant treatment.

Embodiments of the invention also provide for systems (and computer readable media for causing computer systems) to perform the method(s) of the invention. In one embodiment of the invention, there is provided a system for obtaining data from at least one test sample obtained from at least one individual, the system comprising:
a determination module configured to receive at least one test sample and perform at least one test analysis on the test sample to assay for expression of at least one genes (or proteins encoded by those genes) selected from the group consisting of ITGB4 and SNAI2, optionally,
a storage system for storing expression data generated by the determination module; and a display module for displaying a content based in part on the data output from said determination module, wherein the content comprises a signal indicative of the expression of the at least one genes.

However, it should be understood that the modules/systems need not correspond to discreet blocks of code and the described functions can be carried out by the execution of various code portions stored on various media and executed at various times. Furthermore, it should be appreciated that the modules/systems may perform other functions, thus the modules are not limited to having any particular functions or set of functions.

In one embodiment, the system comprises a correlation module for correlating the expression data of the at least one genes (or proteins encoded by those genes) from the determination module, wherein the expression data of each gene (or a protein encoded by the gene) is compared with a reference value or control for the gene (or a protein encoded by the gene) to determine increased or decreased expression of the gene (or a protein encoded by the gene). The display module displays a content based in part on the data from the correlation system, the content optionally comprising a signal indicative of the result. The comparison module may be configured using existing commercially-available or freely-available software for comparing patterns, staining, and may be optimized for particular data comparisons that are conducted.

The “comparison module” can use a variety of available software programs and formats for the comparison operative to compare ITGB4 and/or SNAI2expression information data determined in the determination system to reference samples and/or stored reference data. In one embodiment, the comparison module is configured to use pattern recognition techniques to compare information from one or more entries to one or more reference data patterns. The comparison module may be configured using existing commercially-available or freely-available software for comparing patterns, staining, and may be optimized for particular data comparisons that are conducted. The comparison module provides computer readable information related to the ITGB4 and/or SNAI2 expression levels of the sample.

Suitably, the determination system may be selected from an immunohistochemical detection apparatus, a Western Blot, a Northern Blot, a Southern Blot, quantitative polymerase chain reaction (PCR), reverse transcriptase PCR (RT-PCR), quantitative real time RT-PCR (qRT20 PCR), an enzyme-linked immunosorbent assay (ELISA), protein determination on polyacrylamide gels, and such methods known to those skilled in the art.

Ideally, the system is PCT, or RT-PCR.

In one embodiment of the invention, the content based on the comparison result, or the determination system is displayed on a computer monitor. In one embodiment of the invention, the content based on the comparison result or determination system is displayed through printable media. The display module can be any suitable device configured to receive from a computer and display computer readable information to a user. Non-limiting examples include, for example, general-purpose computers such as those based on Intel PENTIUM-type processor, 30 Motorola PowerPC, Sun UltraSPARC, Hewlett-Packard PA-RISC processors, any of a variety of processors available from Advanced Micro Devices (AMD) of Sunnyvale, California, or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the invention can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The determination system has computer executable instructions to provide e.g., ITGB4 and/or SNAI2 expression levels in computer readable form. The determination system can comprise any system for assaying a breast cancer tumor sample for expression of ITGB4 and/or SNAI2. Standard procedures such as RT-PCR, may be employed.

The information determined in the determination system can be read by the storage device. As used herein the “storage device” is intended to include any suitable computing or processing apparatus or other device configured or adapted for storing data or information.

Examples of an electronic apparatus suitable for use with the present invention include a stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon nucleic acid sequence information. Such information may be provided in digital form that can be transmitted and read electronically, e.g., via the Internet, on diskette, via USB (universal serial bus) or via any other suitable mode of communication.

The methods described herein therefore provide for systems (and computer readable media for causing computer systems) to perform methods as described in the Statements of Invention above.

Systems and computer readable media described herein are merely illustrative embodiments of the invention for performing methods of diagnosis in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, poisoning or nutritional deficiencies.

As used herein the cancer is selected from the group comprising, but not limited to, breast cancer; multiple myeloma, prostate cancer, glioblastoma, lymphoma, fibrosarcoma; colon carcinoma; pancreatic cancer; ovarian cancer; squamous cell carcinoma; basal cell carcinoma; adenocarcinoma; sebaceous gland carcinoma; papillary carcinoma; papillary adenocarcinomas; bronchogenic carcinoma; renal cell carcinoma; hepatoma; bile duct carcinoma; embryonal carcinoma; Wilms' tumour; cervical cancer; uterine cancer; testicular tumour; lung carcinoma; small cell lung carcinoma; bladder carcinoma; epithelial carcinoma; glioma, melanoma; retinoblastoma; and leukaemia. Suitably, the cancer is a solid tumour.

As used herein, the term “treatment” or “treating” refer to an intervention (e.g. the administration of an agent to a subject) which cures, ameliorates or lessens the symptoms of a disease or removes (or lessens the impact of) its cause(s). In this case, the term is used synonymously with the term “therapy”. It can be manifested by a permanent or temporary improvement in the subject's condition. In this context it includes limiting and/or reversing disease progression.

As used herein the terms “prevention” or “preventing” refer to an intervention (e.g., the administration of an agent to a subject), which prevents or delays the onset or progression of a disease, e.g., breast cancer, or the severity of a disease, in a subject, or reduces (or eradicates) its incidence within a treated population.

A “subject in need thereof” refers to a person who has cancer, suspected of having or developing cancer. Preferably, the subject is a person who has breast cancer, suspected of having or developing breast cancer. Preferably the subject is a human and typically a female human.

A “lymph node” is a small organ of the lymphatic system and the adaptive immune system. The lymph nodes are linked in the body by the lymphatic system. There are clusters of lymph nodes near the collarbone and breastbone. The lymph nodes that are closest to the breast are called sentinel lymph nodes and the nodes under the armpit are called axillary lymph nodes.

As used herein “lymph node involvement” refers a lymph node having a cancer cell. It may be also be called “lymph node positive (LN+). This may also be referred to as lymph node metastasis.

As used herein “lymph node negative (LN−)” refers to a lymph node free, or clear, of a cancer cell.

As used herein “breast cancer subject” or “patient” means a subject that has breast cancer, typically a subject who has a primary breast cancer tumour and awaits assessment/stage and/or treatment for the cancer or has already undergone or is undergoing treatment for the primary tumour. The term should also be understood to include a patient or subject who has had a primary breast cancer and is in remission, for example remission following treatment including one or more of tumour resection, first line chemotherapy, or both. Typically, the subject is a female subject. In an embodiment, the subject may be a male subject.

The “breast cancer” is preferably a hormone receptor (HR) positive breast cancer, including tumours that are estrogen receptor (ER) positive and/or progesterone positive (PR). The breast cancer may be HER positive breast cancer (HER+). The breast cancer may also include tumours that are ER+, PR+ and HER+. The breast cancer may also be a hormone receptor negative breast cancer, or a triple negative breast cancer, i.e., without ER, PR or overexpression of HER2.

In one embodiment, the cancer is an early-stage cancer, such as stage I or Il.

The term “control” refers to a value that can be compared with the patient expression. It may be a predetermined value. It is the gene expression levels of the same gene in a particular patient or subject population. It may be a control predetermined from one or more healthy subjects, e.g., an individual or cohort of individuals without breast cancer. The control may also include tissue taken from another part of the breast of the same subject and pathologically defined as non-cancerous (as defined by a pathologist) or from a non-cancerous breast of the same subject. It is within the remit of a person skilled in the art to determine the appropriate control value.

The term “assaying” should be understood to mean quantitative detection of one or more of genes or proteins in the sample. Suitable methods will be known to a person skilled in the art, and include quantitative PCR (qPCR) and hybridisation assays, or sequencing. This includes methods to determine differential expression of a gene (or protein). It includes methods to determine increased and decreased expression of a gene (or protein).

The terms “normal expression” or “moderate expression” as applied to a gene or protein should be understood to mean a level of expression of the gene (or protein encoded by that gene) that is equivalent to a level of expression of the same gene (or protein encoded by that same gene) found in a matched subject or cohort of matched subjects that do not have cancer, i.e. healthy individuals. In this context it may also include the level of expression of the same gene (or protein encoded by that same gene) found in a non-cancerous breast in the same subject or in tissue taken from another part of the breast of the same subject and pathologically defined as non-cancerous (as defined by a pathologist).

The terms “increased expression” as applied to a gene or protein should be understood to mean a level of expression of the gene, or protein, that is higher than the level of the same gene or protein, found in a matched subject or cohort of matched subjects that do not have cancer, i.e., greater than normal expression.

The terms “decreased expression” as applied to a gene or protein should be understood to mean a level of expression of the gene, or protein, that is lower than the level of the same gene or protein found in a matched subject or cohort of matched subjects that do not have cancer, i.e., greater than normal expression. It may be no expression.

The level of expression need not be an absolute value but may rather be a normalised expression value or a relative value. For example, the level of expression can be normalised against housekeeping or reference gene expression.

Methods to determine increased expression are known in the art and it will be understood that all are incorporated herein. Suitably, the determination means or method may be selected from an immunohistochemical detection apparatus, a Western Blot, a Northern Blot, a Southern Blot, quantitative polymerase chain reaction (qPCR), reverse transcriptase PCR (RT-PCR), quantitative real time RT-PCR (qRT-PCR), an enzyme-linked immunosorbent assay (ELISA), protein determination on polyacrylamide gels, immunohistochemical detection apparatus and such methods known to those skilled in the art.

Ideally, the determination system comprises PCR, most typically quantitative polymerase chain reaction (qPCR).

The method used to set thresholds or cut-offs differs depending on the type of analysis used. For qPCR and protein expression it is set at specific points. A sample with altered expression is one falling below or above set values. For example, in a genetic quantification by polymerase chain reaction (PCR) Ct value “cut-off” would be used to determine lymph node status. A PCR threshold is used to determine the Ct value. A higher expression will result in lower values (Ct (cycle threshold) or Cq (quantitation cycle)).

The method of the invention may involve one or more housekeeping genes. Housekeeping, or control genes, are used to normalise the genes or mRNA level of the gene of interest before comparison real time PCR. The housekeeping genes are known to a person skilled in the art and all are incorporated herein. Examples include but are not limited to ACTB, GAPDH, YWHAZ and 18S.

In the specification, the term “neoadjuvant therapy” should be understood to mean treatment given before primary treatment. Primary treatment is generally surgery. Neoadjuvant therapies are generally selected from chemotherapy, hormonal therapy, targeted therapy, radiation therapy, immunotherapy or a combination thereof.

In the specification, the term “adjuvant therapy” should be understood to mean any treatment given after primary treatment. Adjuvant therapy are generally selected from chemotherapy, hormonal therapy, targeted therapy, radiation therapy, immunotherapy or a combination thereof.

In one embodiment, the therapy can be a combination of neoadjuvant and adjuvant therapy.

In the specification, the term “distant metastasis” refers to metastasis or spread of cancer to other organs of the body.

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### EXAMPLES

### Materials and Methods

### Tissue Collection

Tissue samples were collected under ethical approval granted by the University Hospital of Limerick's Ethics Committee and was allocated the identification numbers 22/14 and 141/12. All patients enrolled were informed of their rights, agreed to participate and gave written consent. The operating surgeon extracted tumours core biopsies from the resected tumour mass and samples were stored in Allprotect Tissue Reagent (Qiagen) at −80 ° C.

### RNA Extraction

RNA was extracted from breast tissue samples by initially crushing samples into a fine powder using liquid nitrogen, followed by the RNeasy Lipid Tissue Mini Kit (Qiagen). RNA yields were increased and contaminants minimised through the use of Maxtract tubes (Qiagen) for the initial extraction step. RNA was quantified using a Nanodrop Spectrophotometer (Thermo scientific) and purity was evaluated using a 1% agarose gel to visualise the 28S:18S RNA ratio, all RNA was stored at −80° C. Using the Superscript Vilo cDNA synthesis kit (Invitrogen) 500ng of total RNA was synthesised into cDNA and stored at −20° C.

### RT-qPCR

Real time PCR was performed out using TaqMan gene expression assay kits (Thermo Fisher) and the Quant Studio 7 flex real time PCR system (Thermo Fisher). Firstly, a panel of seven housekeeping genes were evaluated across a range of breast cancer tissue types and the top three most stable genes were identified by using excel NormFinder, these three genes were used for normalisation of each sample (See data Table 4). The expression of 30 genes were analysed in a range of both malignant and non-malignant breast tissue samples. Relative fold change in gene expression was calculated using the 2^{-ΔΔCT} method.

### Statistical Analysis of Test and Validation Datasets:

Test Dataset (n=22)

A descriptive analysis of genes differentially expressed between subgroups of clinical categories was carried out using graphical and numerical methods. These subgroups included tumour grade and lymph node status. Non-parametric tests were used to compare median expression across groups. Spearman's correlation coefficient was used to measure the association between genes expressed. A binary logistic regression analysis was used to classify lymph node status using clinical and gene expression variables. The final model was chosen based on the discriminative ability of the model, which was measured using the C-statistic and the percentage of patients correctly classified. Odds ratios (OR) and 95% confidence intervals (95% CI) for predictor variables are presented. All statistical analysis was carried out using SPSS statistical software package 25. A 5% level of significance was used for all tests.

Validation Dataset (n=67)

Dataset GSE42568 was downloaded from the gene expression omnibus repository (GEO), the dataset was collected using Affymetrix Human Genome U133 Plus 2.0 Array.and affymetrix gene expression analysis was imported as excel file using GEOR (http://www.ncbi.nlm.nih.gov/geo). Raw data was pre-processed and expression was downloaded as log2 normalised. The gene expression from 67 samples identified as estrogen receptor positive (ER+) were then used for analysis. This dataset contained 17 non-malignant tumour samples and 104 malignant samples, 59 of which were node positive, and 45 of which were node negative. In the dataset the only receptor status available was for the estrogen receptor, and so we selected only those who were ER+ (n=67). A binary logistic regression analysis was used to classify lymph node status using the predictor variables identified in the final model for the test dataset. The discriminative ability of the model was measured using the C-statistic and the percentage of patients correctly classified.

### Analysis of Distant Metastasis Free Survival

KMplotter was used to analyse high and low expression of both the ITGB4 and SNAI2 genes (http://www.ncbi.nlm.nih.gov/geo). This repository collects both gene expression and clinical data downloaded from GEO, EGA and TCGA databases. The outcome of interest was distant metastasis free survival (DMFS) measured as time in months from surgery to identified metastasis. Upper/lower quartiles of expression were determined using the best cut off function and used to categorise samples into two groups (high/low expression). DMFS was compared across these groups using Kaplan-Meier plots and the log-rank test.

### RESULTS

### Gene Dysregulation Among Breast Cancer Patients With and Without the Presence of Node Involvement

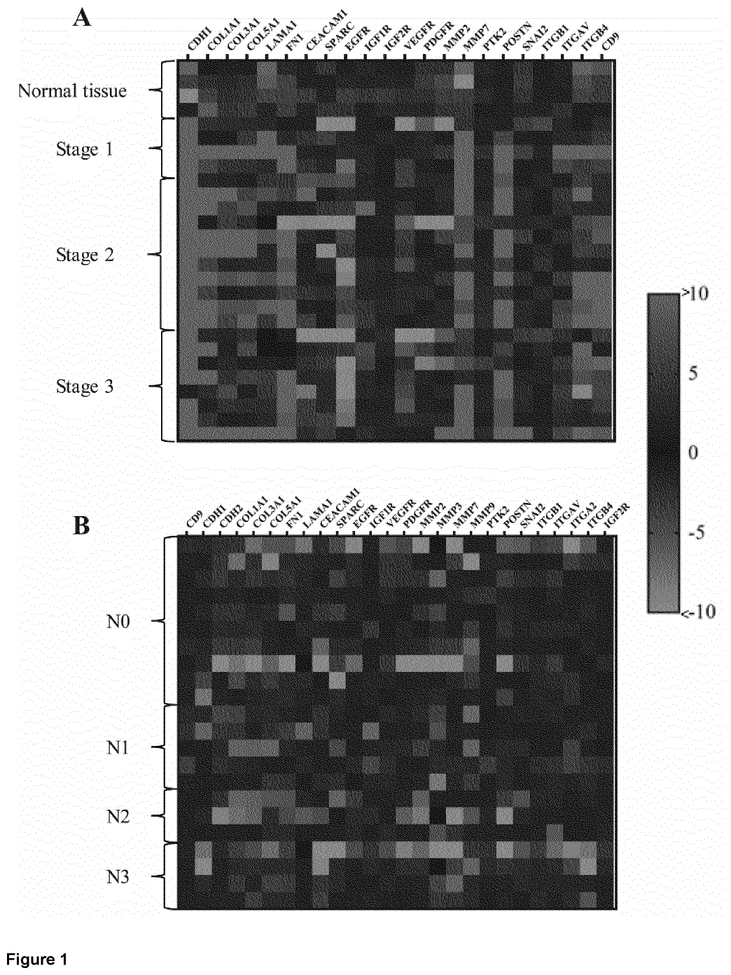

Previously, using a variety of breast cancer cell lines the inventors have shown that components of the ECM induced dysregulation of a selected gene panel (Nolan, J., et al., Collagen and fibronectin promote an aggressive cancer phenotype in breast cancer cells but drive autonomous gene expression patterns. Gene, 2020: p. 145024). The current study aimed to investigate if it was possible to exploit these patterns of dysregulation in breast cancer tissue samples with the overall aim of predicting lymph node involvement. The selected gene panel was then applied to a range of both malignant and non-malignant breast tissues. A panel of seven reference genes were analysed across a range of breast tissue samples (n=25) to determine the three most consistently stable genes across a variety of breast cancer subtypes. The inventors achieved this by using the Normfinder software (see Table 4) and this validation of reference genes was needed to ensure accurate normalisation of data. Using four non-malignant tissue samples, the inventors took the mean expression value of each gene to account for interpersonal variation among the population. Samples of breast tumour tissue were then compared to this mean using the delta-delta ct calculation. The product of this approach was a fold change value allowing the inventors to observe the gene dysregulation which existed between malignant and non-malignant breast tissue (FIG. 1 A).

The aim was to identify key patterns of gene expression which may be used to identify advanced breast cancer. Patients who were lymph node positive and lymph node negative were analysed. Using malignant tissue samples from patients who were pathologically lymph node negative (n=10), the mean value of expression for every gene was taken. Tumour tissue samples from pathologically diagnosed lymph node positive patients (n=12) were compared to this mean value using the delta-delta ct calculation. This approach allowed the inventors to compare and contrast similarities and differences in gene expression between pathologically defined lymph node negative patients and lymph node positive patients (FIG. 1 B).

### Creating a Predictive Model to Identify Lymph Node Positive Breast Cancer Patients

Patient characteristics are given in Table 3.

A descriptive analysis of parameters typically identifiable of progressive cancer e.g. differential gene expression between stage 2 and stage 3, between grade 2 and grade 3 and between those with and without node involvement, was carried out on the test dataset to 5 identify a subset of potential genes for inclusion in a regression model (Table 1).

TABLE 1
Statistical analysis of breast cancer tissue samples from a locally
collected cohort - test dataset (n = 22): Student T-test were
used to determine what genes were expressed differentially between
the following categories: Node: Non-parametric independent t-test
carried out between LN+ and LN−. Grade: Non-parametric independent
t-test carried out between grade 2 and grade 3. Stage: Non-parametric
independent t-test carried out between stage 2 and stage 3.

| Gene | Node | Grade | Stage |
|---|---|---|---|
| CD9 | 1 | 0.149 | 0.54 |
| CDH1 | 0.67 | 0.319 | 0.054 |
| CDH2 | 1 | 1 | 0.375 |
| COL1A1 | 1 | 0.62 | 0.375 |
| COL3A1 | 1 | 1 | 0.375 |
| COL5A1 | 0.67 | 1 | 0.349 |
| FN1 | 1 | 1 | 0.89 |
| LAMA1 | 1 | 0.319 | 0.349 |
| CEACAM1 | 1 | 0.264 | 0.54 |
| SPARC | 1 | 0.621 | 0.89 |
| EGFR | 0.67 | 0.149 | 0.89 |
| IGF1R | 0.67 | 0.621 | 0.349 |
| VEGFR | 0.67 | 0.319 | 0.89 |
| PDFGR | 1 | 0.149 | 0.89 |
| MMP2 | 1 | 1 | 0.375 |
| MMP3 | 0.198 | 0.149 | 0.084 |
| MMP7 | 0.67 | 0.319 | 0.54 |
| MMP9 | 1 | 0.084 | 0.54 |
| PTK2 | 1 | 0.621 | 0.54 |
| POSTN | 1 | 1 | 0.89 |
| SNAI2 | 0.67 | 0.621 | 0.54 |
| ITGB1 | 1 | 0.319 | 0.89 |
| ITGAV | 1 | 1 | 0.349 |
| ITGA2 | 0.67 | 0.264 | 0.375 |
| ITGB4 | 0.198 | 1 | 0.375 |
| IGF2R | 1 | 0.319 | 0.04 |

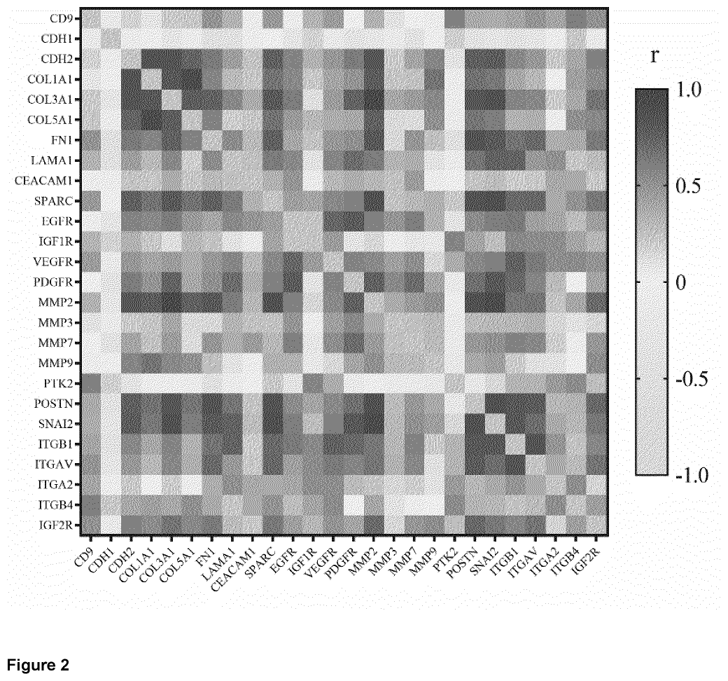

These were correlated with each other using Spearman's correlation coefficient to identify those with potentially independent information (FIG. 2). A binary logistic regression analysis was used to classify lymph node status using clinical and gene expression variables. The final model was chosen based on the discriminative ability of the model, which was measured using the C-statistic and the percentage of patients whose lymph node status was correctly classified. An iterative model building process was used to build a model with the aim of identifying what genes influenced discriminative ability for lymph node status. The initial model included tumour grade and correctly classified lymph node status of 14/22 (63%) patients. The addition of the genes ITGB4 and SNAI2 increased the percentage correctly classified to 73% in the final model (Table 2 A). Increasing expression of ITGB4 was associated with a decrease in the odds of lymph node positive status (OR: 0.53, 95% CI: 0.28%-1.01%). Increasing expression of SNAI2 was associated with an increase in the odds of lymph node positive status (OR: 1.7, 95% CI: 1.0-3.12) (Table 2 B).

TABLE 2
Binary regression analysis on the test population (n = 22)
shows increased discriminative ability in identifying lymph node
positive patients with the addition of grade, SNAI2 and ITGB4.
A

| | | | Percentage |
|---|---|---|---|
| Node | No | Yes | Correct |
| No | 7 | 3 | 70.0 |
| Yes | 3 | 9 | 75.0 |

| | |
|---|---|
| Overall Percentage | 73.0 |

B

| | | Regression | | Odds | 95% CI | 95% CI | |
|---|---|---|---|---|---|---|---|
| | Variable | Coefficient | P-value | ratio | lower | upper | R2 |
| Test | Grade | | 0.376 | | | | |
| Population | Grade 1 | −21 | 0.99 | 0.00 | 0.00 | | |
| | Grade 2 | 2 | 0.162 | 8.0 | 0.433 | 149.8 | 0.55 |
| | SNAI2 | 0.56 | 0.05 | 1.7 | 1.00 | 3.12 | |
| | ITGB4 | −0.62 | 0.05 | 0.53 | 0.280 | 1.01 | |

Overall discriminative ability from a binary regression analysis of locally collected patient tissue samples, predicting node involvement through the use of three variables.

TABLE 3
Comparison of patient characteristics and binary regression
analysis between test and validation datasets.
A
Patient Characteristics

| | | 1 | | 2 | 3 | |
|---|---|---|---|---|---|---|
| Grade | Test population | 2 | (9%) | 14 (64%) | 6 | (27%) |
| | Validation population | 11 | (11%) | 40 (38%) | 53 | (51%) |
| Tumour Stage | Test population | 5 | (23%) | 15 (68%) | 2 | (9%) |
| | Validation population | 35 | (34%) | 66 (63%) | 3 | (3%) |

| | | Positive | | Negative |
|---|---|---|---|---|
| Node Stage | Test population | 12 | (55%) | 10 (10%) |
| | Validation population | 59 | (57%) | 45 (43%) |

| | | Average | Node− | Node+ | Min | Max |
|---|---|---|---|---|---|---|
| Age | Test population | 58 | 58 | 59 | 41 | 77 |
| | Validation population | 58 | 61 | 56 | 31 | 89 |

B

| | | | | Percentage |
|---|---|---|---|---|
| | Node | No | Yes | Correct |
| Test population | No | 7 | 3 | 70 |
| | Yes | 3 | 9 | 75 |
| | Overall percentage | | | 73 |
| Validation population | No | 19 | 10 | 66 |
| | Yes | 8 | 30 | 79 |

| | |
|---|---|
| Overall percentage | 73 |

A) Patient characteristics across both the test and validation datasets. Including grade, tumour and node stage and age (mean average) B) Overall discriminative ability from a Binary regression analysis predicting node involvement using three variables in our locally collected patient tissue samples and validation in the GSE42568 cohort.

TABLE 4
Normfinder results on breast tissue samples. Normfinder analysis on
25 breast tissue samples was used to determine the most constituently
expressed stable genes across a range on non-malignant and malignant
breast tissue samples from a range of breast cancer subtypes.

| Gene Name | Stability value | Best Gene |
|---|---|---|
| ACTB | 1.023 | HRPT1 |
| PGK1 | 0.435 | |
| PPIA | 0.277 | |
| HRPT1 | 0.276 | |
| GUSB | 0.686 | |
| GAPDH | 1.477 | |
| TBP | 1.751 | |

### Validation of the Model

Following this, the patient numbers were expanded through the use of an independent dataset downloaded through GEO—GSE42568, also collected in Ireland. Upon close examination of the patient characteristics (Table 3 A), there is similarity among the test and validation datasets across tumour and node staging, tumour grade, and age. This dataset contained 17 non-malignant tumour samples and 104 malignant samples, 59 of which were node positive, and 45 of which were node negative. In the dataset the only receptor status available was for the estrogen receptor, and only those who were ER+ (n=67) were selected. A binary logistic regression analysis was used to classify lymph node status using the predictor variables identified in the final model for the test dataset. The discriminative ability of the model was measured using the C-statistic and the percentage of patients correctly classified. When a model was fit to the GSE42568 dataset using the same predictor variables from the final test model, the discriminative ability of the model is similar with lymph node status correctly classified for 49/67 (73%) patients and an increase from 65% with just tumour grade included as a predictor variable (Table 3 B).

### Analysing the Expression of ITGB4 and SNAI2 in Distant Metastasis Free Survival

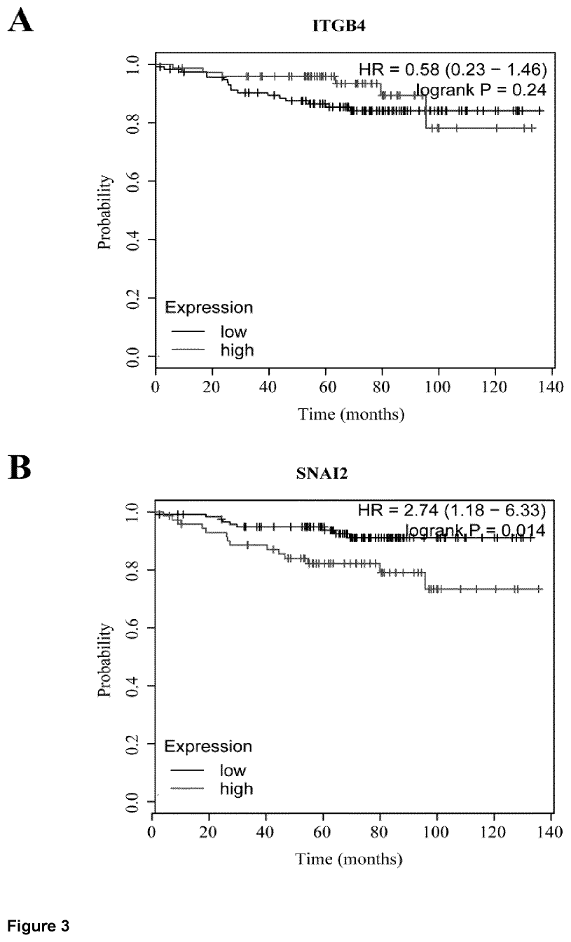

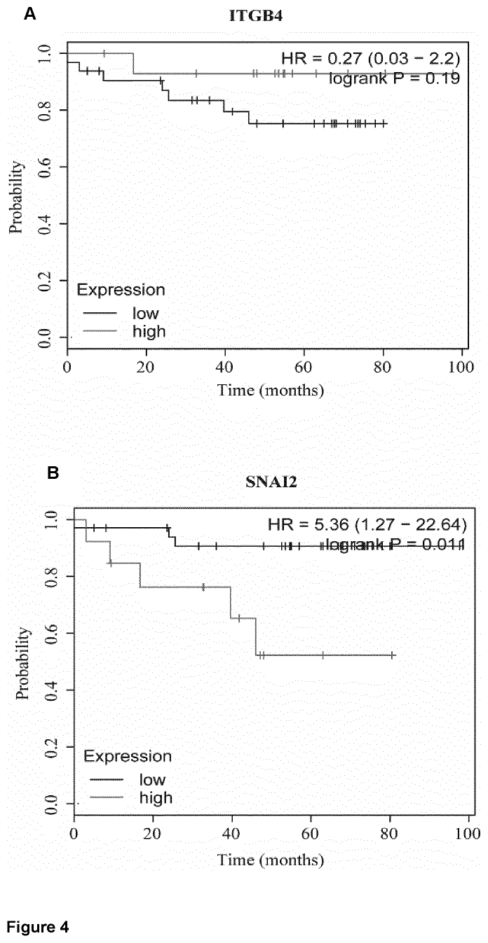

To support this result, a KMPLot.com was utilised to investigate the expression of ITGB4 and SNAI2 in a larger cohort by assessing distant metastasis free survival (time from surgery to time of identified metastasis). This database assembles gene expression and survival information from GEO, EGA and TCGA datasets that use Affymetrix HGU133A and HGU133 Plus 2.0 microarrays. The patient population was selected according to the presence of ER and progesterone receptor (PR) and further included both HER2 positive and negative samples (n=191). Patients were grouped according to high or low expression of each gene and Kaplan-Meier plots used to compare survival across groups (FIG. 3). The population was further narrowed by selecting patients who were ER/PR/HER2 positive (n=47) (FIG. 4). Interestingly the analysis adds to the results from the regression model on the role of SNAI2. Those with high SNAI2 had a poorer DMFS prognosis than those with low SNAI2 expression (p-value from log-rank test=0.01, FIG. 3). It can be seen that those with a low expression of ITGB4 have a worse DMFS between 30-90 months. However, those with high expression of ITGB4 begin to display a poorer rate of DMFS between 90-140 months. Notably, the role of low ITGB4 is further compounded through the analysis of hormone receptor positive patients (FIG. 4). Although it should be noted that the patient dataset is smaller (n=47), it appears those with low ITGB4 expression have a worse DMFS prognosis when compared to those with high ITGB4 expression (p-value from log-rank test=0.19).

### DISCUSSION

The current inventors have shown that there is dysregulation among the expression of a selected gene panel when comparing malignant and non-malignant breast tissue. The inventors have further demonstrated that differences in gene expression exist between patients who are LN+ and LN−. Using non-parametric t-tests the inventors further examined the differences in expression of each gene among categories that relate to cancer progression clinically, allowing the inventors to narrow down a subset of genes which displayed dysregulation between these categories. Additionally, the inventors performed Spearman's correlations to determine what genes showed similar patterns of expression and hypothesised that any genes which were correlated would provide similar information and therefore may not add any independent discriminative ability to the model. The inventors were limited in the number of predictor variables to be included in the final model by the small sample size (n=22). They built a model by identifying if any combination of genes could maximise discrimination between LN+ and LN− patients. The combination of grade, ITGB4 and SNAI2 correctly classified the lymph node status of 73% of patients, with a downregulation of ITGB4 and upregulation of SNAI2 increasing the odds of positive lymph node status. The inventors used these predictors on a dataset downloaded from GEO—(GSE42568), also collected in Ireland. The percentage of patients with correctly classified node status by the model was similar to that of the test dataset (73%). To further investigate these patterns the inventors investigated this by analysing DMFS among breast cancer patients on KMplot.com.

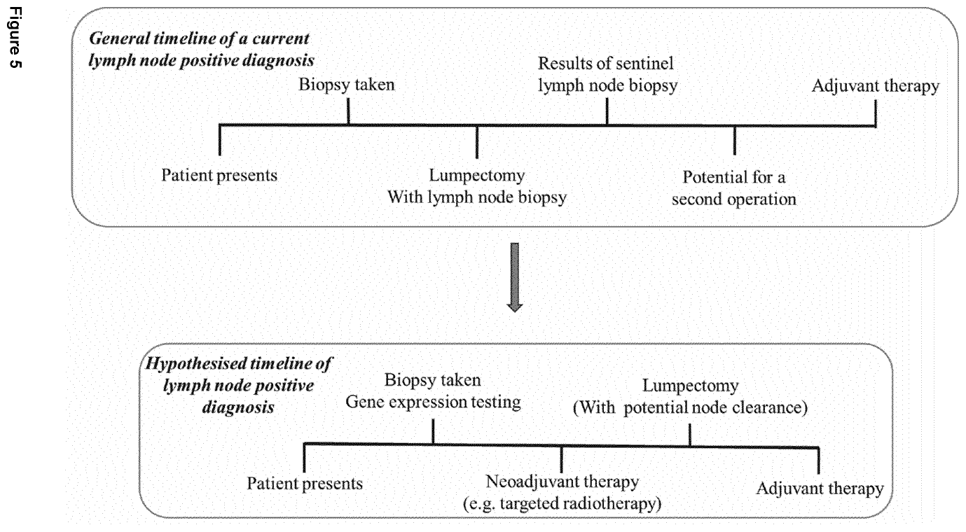

To add to the findings, the inventors were able to replicate the study in a larger independent patient cohort, thus validating our results. Using the GSE42568 publicly available dataset, also collected in Ireland, the initial findings were validated. This combination of ITGB4 and SNAI2 as predictors of lymph node involvement and of breast cancer progression are to the best of our knowledge unique. There is a current lack of prospective gene expression tools for the identification of lymph node involvement. By preoperatively identifying lymph node involvement, this allows for a more accurate identification of patients who require lymph node sampling. This also unlocks the prospect of more accurately identifying those who would benefit from neoadjuvant radiotherapy to the axilla in a bid to prevent further migration, and, ultimately protect patients from unnecessary sentinel lymph node surgery (FIG. 5). Ultimately, more clinically relevant gene expression tools allow clinicians to make decisions that are more informed when considering therapy plans and follow-up practices, with huge potential for improving patient outcomes and quality of life.

1. A method for determining the presence or absence of lymph node metastasis in a breast cancer subject, the method comprising,
assaying for expression of genes (or proteins encoded by the genes) ITGB4 and SNAI2 in a sample from said subject,
wherein an increased expression of ITGB4 and a decreased expression of SNAI2 compared with the expression of the same genes (or proteins encoded by said genes) in a subject without cancer is indicative of the absence of lymph node metastasis in said subject, or,
wherein an increased expression of SNAI2 and a decreased expression of ITGB4 compared with the expression of the same genes (or proteins encoded by said genes) in a subject without cancer is indicative of the presence of lymph node metastasis in said subject.

2. The method of claim 1, wherein the sample is a breast tumour biopsy sample.

3. The method of any one of the preceding claims, wherein the lymph node is selected from one or more of a sentinel lymph node and an axillary lymph node.

4. The method of any one of the preceding claims, wherein said breast cancer is selected from the group comprising a hormone receptor (HR) positive breast cancer, including tumours that are estrogen receptor (ER) positive and/or progesterone positive (PR), a HER positive breast cancer (HER+), a hormone receptor negative breast cancer and a triple negative breast cancer.

5. The method of claim 4, wherein the breast cancer is a hormone receptor (HR) positive breast cancer.

6. The method of any one of the preceding claims, wherein the subject is awaiting assessment and has not undergone any treatment.

7. The method of any one of the preceding claims, wherein the sample is from a primary breast tumour.

8. The method of any one of the preceding claims, wherein the method further comprises determining the grade of tumour present in the subject.

9. The method of any one of the preceding claims, wherein said increased expression of SNAI2 is 1.5 fold or more, and wherein said decreased expression of ITGB4 is 1.5 fold or more.

10. The method of any one of claims 1 to 8, wherein said increased expression of ITGB4 is 1.5 fold or more, and wherein said decreased expression of SNAI2 is 1.5 fold or more.

11. A method for predicting risk of distant metastasis in a subject with breast cancer, the method comprising, assaying for expression of genes (or proteins encoded by the genes) ITGB4 and SNAI2 in a sample from said subject,
wherein an increased expression of ITGB4 and a decreased expression of SNAI2 compared with the expression of the same genes (or proteins encoded by said genes) in a subject without cancer is indicative of the absence of lymph node metastasis in said subject and a low risk of distant metastasis in said subject, or
wherein an increased expression of SNAI2 and a decreased expression of ITGB4 compared with the expression of the same genes (or proteins encoded by said genes) in a subject without cancer is indicative of the presence of lymph node metastasis in said subject and a high risk of distant metastasis in said subject.

12. A method for identifying a subject with breast cancer suitable for treatment, said method comprising determining the presence or absence of lymph node metastasis in said subject according to the method of any one of claims 1 to 10, and determining the treatment plan based on the determination.

13. The method of claim 12, wherein said treatment is neoadjuvant therapy.

14. The method of claim 12 or 13, wherein said treatment is lymph node sampling or dissection to confirm the presence of lymph node metastasis in the subject and/or to determine the extent of involvement.

15. The method of claim 14, wherein said treatment further comprises neoadjuvant therapy and surgery.

16. A method for staging a breast cancer in a subject, said method comprising determining the presence or absence of lymph node metastasis in said subject according to the method of any one of claims 1 to 10, and determining the stage of a cancer based on the presence or absence of lymph node metastasis in the subject.

17. A system for obtaining data from at least one test sample obtained from at least one subject, the system comprising:
a determination module configured to receive at least one test sample and perform at least one test analysis on the test sample to assay for expression of at least one gene (or proteins encoded by the gene(s)) selected from the group consisting of ITGB4 and SNAI2, optionally,
a storage system for storing expression data generated by the determination module; and
a display module for displaying a content based in part on the data output from said determination module, wherein the content comprises a signal indicative of the expression of the at least one gene.

18. A method for treating breast cancer in a breast cancer subject, said method comprising determining the presence or absence of lymph node metastasis in said subject according to the method of any one of claims 1 to 10, classifying said subject as having lymph node metastasis or absence of lymph node metastasis and administering cancer treatment to said subject based on the classification.

19. A method for determining a treatment plan for a subject with breast cancer said method comprising determining the presence or absence of lymph node metastasis in said subject according to the method of any one of claims 1 to 10, classifying said subject as having lymph node metastasis or absence of lymph node metastasis and determining the treatment plan based on the classification.

20. The method of claim 18 or 19, wherein the treatment is neo-adjuvant treatment.

21. The method of claim 18 or 19, wherein the treatment is a surgical treatment to remove the tumour or part thereof and/or lymph nodes, or part thereof.

22. The method of claim 20, wherein the subject subsequently undergoes surgical treatment to remove the tumour or part thereof and/or lymph nodes or part thereof.
